(19) United States
(12) Patent Application Publication
(10) Pub. No.: US 2002/0187203 A1
(43) Pub. Date: Dec. 12, 2002

(54) Stable antimicrobials in structured water

(76) Inventors: Gheorghe Cioca, Lake Grove, NY (US); Mirela Cristina Ionita-Manzatu, Old Bethpage, NY (US); Steven F. Schnittger, Huntington Station, NY (US); Vasile Ionita-Manzatu, Old Bethpage, NY (US); Andrew J. Bevacqua, East Setauket, NY (US)

Correspondence Address: KENYON & KENYON, ONE BROADWAY, NEW YORK, NY, 10004, US

(21) Appl. No.: 09/838,649

(22) Filed: Apr. 19, 2001

(51) Int. Cl. A61K 33/00; A61K 33/38; A61K 31/19

(52) U.S. Cl. 424/600; 424/618; 514/574

(57) ABSTRACT

The invention relates to structured water and the ability to achieve antimicrobial activity when the structured water has two antimicrobial agents in its cluster structure. One of the two antimicrobial agents can be stabilizing agent for the other antimicrobial agent. Thus, the antimicrobial structured water contains a combination of positively charged ions having antimicrobial activity and a stabilizing agent present within its cluster structure. In particular, the present invention relates to potassium sorbate and ionic silver incorporated within the cluster structure of either electropositive (S water) or electronegative (I water). The structured water, having the stabilizing agent and the positively charged ions incorporated within the cluster structure of structured water acts as a preservative when used in cosmetic or pharmaceutical compositions. The structured water of the present invention also has a stabilizing effect on the antimicrobial activity of the ionic silver. The present invention also includes methods of treating, ceasing or retarding the onset of skin diseases associated with microbes, and thereby, promotes the overall general health of the skin.

### FIELD OF THE INVENTION

The present invention relates to antimicrobial structured water and compositions containing antimicrobial structured water. In particular, the invention relates to the cluster structures of structured water containing silver ions, and the stabilization of the antimicrobial activity of silver ions by being present within the cluster structure of structured water.

### BACKGROUND OF THE INVENTION

Silver complex compounds and colloidal silver are believed to have therapeutic and antibacterial activity. Since about the time of the Vikings (800 A.D.), silver has been heralded for its bactericidal activity. In ancient times, for example, there was the belief that disease could not be transmitted by drinking from a silver cup. Today, eating utensils are still referred to their common name “silverware” even though their actual composition is typically stainless steel. In the early part of the 20^{th} century, silver was ground into ultrafine particles and suspended in water for therapeutic uses. In this form, it is commonly referred to as colloidal silver and it has been used for infections, diseases, and burns. When antibiotics were developed on a commercial basis, around the end of World War II, the use of silver waned.

Colloidal silver is a suspension of monovalent silver particles in a colloidal base, typically water. The silver particles are positively charged and have a minute particle size, approximately 0.001 to 0.006 microns. The smaller the particle size of the silver, the greater the therapeutic effect of colloidal silver is believed to be. To produce colloidal silver, a small generator emits a small D.C. current through an electrolyte with silver electrodes. A voltage of about 30 V is recommended. Minute molecular sized particles, having a positive electrical charge, are drawn off of the positive electrode. The positive charge of the particles is important to maintain the therapeutic and antibacterial activity of the silver. Numerous ultrafine silver particles, which are positively charged, creates a large force of repulsion among the individual particles and prevents them from agglomerating. However, the electrical charge is unstable and gradually dissipates. The particle size of the silver increases as it loses its positive charge, and produces varying colors. The color of the suspension changes from yellow, to brown, to red, to gray, and finally to black as the particle size grows. Thus, the presence of color indicates that the silver particles are inferior.

Proteins are known to stabilize the silver ionic particles in suspension. The protein increases the viscosity of the colloidal solution and keeps the silver particles in suspension for a longer period of time. However, after time, the silver particles still settle out and the solution must be agitated to redisperse the particles. In addition, the use of stabilizers also has an adverse effect on the beneficial effects of the silver particles themselves. Other alternatives to colloidal silver which have been proposed to avoid the stability problems associated with the monovalent silver ion are, for example, polyvalent forms of silver. In particular, silver (II, III) disinfectants have been reported as having improved activity compared to monovalent silver, and further, specific silver (II, III) compounds have been reported as being stable against photodegradation unlike monovalent silver. “Silver (II, III) Disinfectants” Soap/Cosmetics/Chemical Specialties, March, 1994, pgs. 52 to 59. The use of silver ion salts are also known to be irritating to the skin, and mucous membranes. To protect silver from the light, because it is photosensitive, special packaging requirements must be employed such as the use of dark glass bottles.

The purity of the water in which silver ions are suspended is an important factor in making colloidal silver and also contributes to the size of the silver particles produced. High quality distilled water is preferable. However, the use of structured water has not previously been suggested in relation to the suspension of silver ions for use as an antimicrobial. Developments in water technology have led to the development of structured water, commonly referred to as I and S water. It has been postulated that water, itself, may in fact change structure and function once it has been taken into tissues and cells (see, e.g., Stillinger, F. H., “Water Revisited”, Stillinger, Science, vol. 209: no. 4455, pp. 451-57, 1980). Taking this theory into consideration, the use of I and S structured waters in compositions has increased. For example, several oil-in-water emulsions are disclosed in RO 107546, RO 107545, and RO 107544 using structured water. These compositions relate to the use of structured water in specific cosmetic products, for the treatment of oily skin, dry skin, or acne.

Different biological properties have been suggested for the two types of structured water. S water is said to have a stimulatory effect on enzymatic and other biosynthetic processes; whereas, I water is said to be inhibitory of the same processes. Substantial differences are found among the UV spectra of I, S, tap and deionized waters, particularly in the 200 to 250 nm band. When their reactivities are measured in an electronographic field, I, S and tap waters also show significant differences. In particular, with respect to tap water, the total light flux emitted after electronographic stimulation with a positive impulse, I⁺, is substantially equivalent to its negative impulse, I⁻. For structured water, on the other hand, S water stimulated in the same way exhibits a very high light reactivity to a positive impulse, while its reactivity to a negative impulse is almost equivalent to that of distilled water, yielding a positive to negative ratio of greater than 1. Further in contrast, I water samples show a high light reactivity to a negative impulse, with reactivity to a positive impulse approximately equivalent to distilled water, and having a ratio of positive to negative less than 1.

It is known to add active agents, as separate and individual components, to structured water. In U.S. Pat. No. 6,139,855, for example, I and S waters are described as being able to enhance the level of certain types of actives, including an antioxidant. This result has been observed with materials of very distinct chemical identity and biological activity, particularly, caffeine an anti-irritant, and BHT as an antioxidant. However, these biological actives are in combination with the structured water (i.e., the active is separate from the cluster structures of the structured water). It is also described in U.S. patent application Ser. No. 09/632059 that compounds having antioxidant activity can be incorporated in the cluster structure of structured water.

It has now surprisingly been discovered that structured water is capable of stabilizing the antimicrobial activity of silver ions while maintaining its beneficial effects in combating bacteria, yeast, fungus, and viruses.

### SUMMARY OF THE INVENTION

The present invention relates to structured water comprising cluster structures having at least two antimicrobial agents within its cluster structures, and compositions containing the structured water of the present invention. The antimicrobial activity of the agent arranged within the cluster structure of structured water is stabilized. Specifically, the antimicrobial agents are ionic silver and potassium sorbate because they are particularly suited for incorporation into the cluster structure of structured water. The structured water of the present invention, having the ionic silver and potassium sorbate in its cluster structure, can be added to cosmetic or pharmaceutical compositions in an antimicrobial effective amount, to preserve and protect the composition against microbes. In addition, the compositions can be topically applied to the skin to protect the skin from microbes, and to treat or retard the growth of microbes which increase the likelihood of the onset of skin diseases.

The ionic silver and potassium sorbate are integrated in the cluster structure of structured water by feeding a solution of unstructured feed water containing silver ions and potassium sorbate through a device for producing structured water. The silver particles are added to the feed water before the structured water is produced. Passing the combined silver particles and feed water through the device causes the feed water to divide into fractions of clusters which form the cluster structures of the structured water. The silver ions and potassium sorbate are integrated within the cluster structures. The present invention also includes a method of stabilizing the antimicrobial activity of the ionic silver as the silver ions are protected against agglomeration when they are inside of the cluster structures of the structured water. The surface tension of the feed water is reduced before treating the feed water in the device for producing structured water. In addition, a stabilizing agent, potassium sorbate, can be added to the unstructured and untreated feed water. Because of the ability to protect the skin and its surface from microbes, the structured water compositions of the present invention also aid in promoting the health of the skin.

### DETAILED DESCRIPTION OF THE INVENTION

It has now been discovered that ionic silver and potassium sorbate can be incorporated into the cluster structure of structured water. The silver ions are incorporated in the cluster structure and stabilized by the presence of the potassium sorbate. The resulting structured water has antimicrobial activity and the silver does not precipitate out of the structured water. As noted above, structured water is known in the art. In general, structured water contains electronegative and electropositive clusters of water molecules stabilized by ions. Each of these two types of clusters, when they are present in water, is commonly referred to as “I water” and “S water”. On the one hand, I water contains electronegative clusters of water molecules stabilized by a majority of anions, and conversely, on the other hand, S water contains electropositive clusters of water molecules stabilized by a majority of cations. In each case of I water and S water, cluster structure stabilizing anions are, for example, Cl⁻, PO₄⁻³, SO₄⁻² ions and cluster structure stabilizing cations are, for example, Ca⁺², Mg⁺², Na⁺, K⁺ ions. Interaction of the dipolar molecular structure of feed water containing stabilizing ions with an electrical field simultaneously produces I and S water. In general, the conductivity of I water is characterized by C (μS/cm) of about 900 to 3500, and a pH of about 2.0 to 4.0; and the conductivity of S water is characterized by C (μS/cm) of about 600 to 2500, and a pH of about 10.0 to 12.0.

The structured I water of the present invention having silver ions and potassium sorbate in its cluster structure is characterized by C (μS/cm) of about 1500 to 3000, and a pH of about 2.0 to 3.5. The structured S water of the present invention, with the silver ions and potassium sorbate in its cluster structure has a C (μS/cm) of about 600 to 2000, and a pH of about 10.0 to 13.0. It is believed that I water has less silver ions and potassium sorbate in its cluster structure than S water. The concentration of cluster structure stabilizing cations and anions in the feed water used to produce the structured water affects the stability of the silver ions within the cluster structure of structured water. In addition, if the amount of the silver ions is too great, they will precipitate out because the are not part of the cluster structure. This will be evidenced by, for example, the presence of silver ions that settle out of the structured water. Precipitation of silver is similarly experienced with colloidal silver when it is subjected to normal environmental conditions. Stability of the silver ions in the cluster structure is also enhanced by reducing the surface tension of the feed water. Therefore, the feed water can be pre-treated to reduce its surface tension. When the silver ions and the potassium sorbate are nestled in the cluster structure of structured water, the present invention protects the silver ions from destabilizing factors, such as, for example, light and oxygen. The silver ions and the potassium sorbate are protected while also providing desirable anti-microbial activity. The antimicrobial activity of the structured water of the present invention is stable for years, more specifically about 1 to 5 years.

Although the ions of the ionic component stabilize the cluster structure of structured water, it has been surprisingly discovered by extensive research that the addition of silver ions and potassium sorbate to the feed water causes them to be integrated within the cluster structure when processed in a structured water producing device. The antimicrobial structured water is effective against yeast and bacteria. Specifically, I water is effective against yeast and bacteria, and S water is effective against bacteria. While not wishing to be bound by any particular theory, the antimicrobial activity of structured water is more effective than simple addition of traditional antimicrobials to water. Traditional antimicrobials physically kill microbes by coming into contact with the microbe. However, it is believed that specific wave frequencies of the cluster structure containing the silver ions and potassium sorbate have a fatal effect on the microbes. The synergism of the silver ions, with the other cluster structure stabilizing ions in the cluster structure, upon being incorporated into the cluster structure creates an in-phase oscillation of a particular frequency and wavelength. Traditionally, the antimicrobial itself has to come into physical contact with the microbe, however, with the present invention, it is the frequency of waves that acts on the microbe to bring about its death. The antimicrobial and/or antibacterial activity of the structured water, and as used in the present specification, the terms “antimicrobial and antibacterial activity”, refer to the ability to act as a preservative and the ability to exhibit preservative activity as such is known in the art.

Silver ions incorporated within the cluster structure of structured water have a positive electrical charge, large mass, and large ionic radius. The ability to enrich structured water with silver ions is surprising because of their large ionic radius. The other cluster structure stabilizing ions have a considerably smaller ionic radius than the silver ion. Therefore, there is no room for the silver ion to replace the other stabilizing ions, and the large silver ion is not simply incorporated into the cluster structure of structured water. However, it has been discovered that the potassium ion causes a perturbation in the cluster structure that opens a space for the large silver ion to enter into the cluster structure system.

The silver ions incorporated within the network of the cluster structure, can be added to the feed water as, for example, silver nitrate, silver lactate, silver, and any other water soluble source of silver ion. The concentration of silver ions in the unstructured feed water is about 0.001 to about 1.0 mg/100 ml, preferably 0.01 to 0.5 mg/100 ml, and more preferably about 0.02 to 0.4 mg/100 ml, as measured by atomic absorption analytical methods. The resulting structured water having ionic silver in its cluster structure contains about 0.01 to about 0.5 mg/100 ml of ionic silver. It is believed that some of the silver ions may be lost during filtering and structured water processing. The potassium sorbate, is added to the feed water in an amount of about 10 to 200 mg/100 ml, and preferably 20 to 140 mg/100 ml, potassium sorbate. In the structured water, potassium is present in greater amounts in the S water than in the I water.

The stability of the silver ions in the structured water is also dependent upon reducing the surface tension of the feed water. Thus, the inherent surface tension of the feed water is reduced before treating it with the electrostatic field. Alternatively, water without any stabilizing ions or antimicrobials can be treated to reduce its surface tension (i.e., plain water). Any known method can be used to reduce surface tension of the feed water. However, in the present invention, to reduce the surface tension, preferably, a tourmaline filter is used. Tourmaline, well known as a gem, exhibits an unusual variety of pyroelectric and piezoelectric properties. In the present invention, the filter can take on any shape, but is preferably a cylinder closed at each end by covers which hold inlet and outlet tubes. The filter material inside of the cylinder is made of symmetrical layers of ceramic particles of various sizes. There are at least three particle sizes used in the filter. The ceramic particles are coated with tourmaline, and each of the layers as well as the ends of the tube are separated by foam and/or sponge.

The feed water is fed through the filter at a flow rate of about 10 to 200 L/hour. Due to the electrostatic fields of tourmaline crystals, dissociation of water occurs and produces H⁺ ions and OH⁻ ions, and finally produces hydronium ions H₃O⁺ and hydrated hydroxyl ion H₃O₂⁻ which act as a surfactant. The tourmaline treated water is ready for use as the feed water to be fed through the structured water making device. Another tourmaline filter suitable for lowering surface tension is described in U.S. Pat. No. 5,770,089, the contents of which are incorporated herein by reference.

Feed water used to make the structured water of the present invention comprises a stabilizing ionic component in addition to the silver ions and potassium sorbate. The stabilizing ionic component supports the cluster structure of the structured water, and therefore, as a consequence, stabilizes the structured water itself. The feed water is an aqueous solution and has a C (μS/cm) of about 350 to about 550 and a pH of about 5.0 to about 7.5. The aqueous solution can be deionized water, distilled water or tap water. Preferably, the water is deionized water. Specifically, the feed water solution is prepared with a cluster structure stabilizing ionic component of extremely small concentrations of cations and anions such as for example, CaCl₂, MgCl₂, Na₂SO₄, KH₂PO₄, and KNO₃. The range of concentrations of ions in the ionic component can be, for example, CaCl₂ in an amount of about 5.00 to 100.00 mg/100 ml of the feed water, MgCl₂ in an amount of about 1.00 to 10.00 mg/100 ml, Na₂SO₄ in an amount of about 2.00 to 90.00 mg/100 ml, KH₂PO₄ in an amount of about 0.20 to about 2.00 mg/100 ml, and KNO₃ in an amount of about 0.90 to 9.00 mg/100 ml. For example, the ion content of the ionic component can be 11.00 mg/100 ml CaCl₂, 4.20 mg/100 ml MgCl₂, 5.00 mg/100 ml Na₂SO₄, 0.70 mg/100 ml KH₂PO₄, and 1.10 mg/100 ml KNO₃. The feed water has, for example, a pH of about 6.0 to 6.4 and a C (μS/cm) of about 470 to 520.

After the desired feed water is prepared, it can be processed to make the structured water. The present invention includes methods of making structured water having positively charged silver ions and potassium sorbate within its cluster structure. The process of making structured water is described for example, in RO 88053 which describes a method for producing “B” or basic (S-type) water, and RO 88054 which discloses a method for making “A” or acid (I-type) water. Improvements in simultaneously making either of these types of water are further described in U.S. Pat. No. 5,846,397. The content of each of these documents is incorporated herein by reference. The structured water making device uses one or several serial structuring cells placed in a chemically inert parallelipipedic column made out of glass or plexiglass, for example.

The cells are typically supported on four legs and are enclosed on top by a cover, but other means of support and enclosure can be used. Each structuring cell has a pair of activators and numerous working spaces. The working spaces are generally arranged such that there are two working spaces available to supply feed water, two working spaces each for generating, and for gathering and disposing S water, and two working spaces each for generating, and for gathering and disposing I water. In the space for generating or producing the S water, the polarization and energy needed for binding water molecules, by hydrogen and hydroxyl bridges, in polymolecular aggregates (i.e., clusters) with radicals (Rₘ⁺ stabilizing ions), is present as a result of the electrostatic field being about 80 to 120 V. Similarly, polymolecular aggregates (i.e., clusters) with radicals (Rₖ⁻ stabilizing ions) are simultaneously formed to make I water, in the space for producing I water.

The activators are made of two inox stainless (e.g., stainless steel) lamellar electrodes and are held tightly in place by a gasket in the parallelipipedic column. The positive electrode is in the space for gathering and disposing the I water and the negative electrode is in the space for S water. The activators which are arranged as a sandwich of chemically inert porous membranes are resistant to solutions having a pH of about 2 to 14, by means of plastic spacing pieces. The feed water passes through the activators. An electrostatic field of about 80 to 120 V is applied between the two electrodes in the structuring cell. The feed water is fed through the parallelipipedic column with a volume, for example, of about 80 to 220 L, at a flow rate of about 100 to 220 L/hour to make structured water having the silver ions and potassium sorbate in its clustered structure.

The structured water of the present invention does not require special storage conditions or special packaging to protect it from destabilizing factors. Further, the cluster structure of structured water is very stable. The potential energy of the system of cluster structures in structured water as a whole is minimized. In addition, the structured water containing ionic silver in its cluster structure does not stain the skin, as colloidal silver is believed to cause argyria, nor does it modify the color of the product in which it is contained. The structured water having small amounts of silver ions and potassium sorbate in its cluster structure has antimicrobial activity better than traditional preservatives such as parabens, and the like. Therefore, the structured water is useful for its antimicrobial properties and can be used in a cosmetic or pharmaceutical composition as a replacement for added preservative compounds. This aids in reducing formulation problems which can occur with added preservatives that may interact with active agents in the formula or other desired features of the formula.

Structured I or S water, or a combination of I and S water having the silver ions and potassium sorbate within its cluster structure, can constitute the entire aqueous component of the composition. Thus, the structured water of the present invention can be used to provide antimicrobial activity in any topical or non-topical cosmetic or pharmaceutical product in which there is an aqueous component. In other words, the structured water of the present invention having the silver ions and potassium sorbate in its cluster structure acts as a preservative. The present invention can be used as a preservative in compositions without any added preservatives. The antimicrobial effective amount of structured water having the silver ion and potassium sorbate in its cluster structure when used in a cosmetic or pharmaceutical composition can be 15.0 to about 99.9 percent by weight of the composition as a whole, more preferably about 15 to 80 percent, and more preferably about 15 to 60 percent.

Use of the term “antimicrobial effective amount” herein means an amount sufficient to prevent, reduce, or cease the growth of microbes and their harmful effects substantially equally to or better than about 0.01 to 0.50 percent, preferably about 0.02 to 0.20 percent paraben, silver and potassium sorbate, or any other known preservatives, added to water and simply comixed. The actual comparable amount varies depending on the traditional antimicrobial being replaced and the microbe being protected against. With respect to “antibacterial effective amount” as used herein the same definition applies as previously defined for antimicrobial effective amount except it applies only to bacteria. In addition, because of its antimicrobial activity, the structured water of the present invention can be used in products to clean tools and utensils such as those used in medical facilities, surgical rooms, manufacturing equipment, and manufacturing areas in an environmentally conscious manner.

The structured water having silver ions and potassium sorbate in its cluster structure can be used in a purely aqueous vehicle, a hydroalcoholic vehicle, or it can be used as part of the aqueous phase of any emulsion such as, for example, a water-in-oil or oil-in-water emulsion to provide antimicrobial activity. The form the vehicle takes can be any which is suitable for topical application to the skin, for example, solutions, colloidal dispersions, emulsions, suspensions, creams, lotions, gels, foams, mousses, sprays and the like. For example, it can be used in skin care products, such as cleansers, toners, moisturizers, masks, scrubs, and the like, and it can be used in makeup products, such as lipsticks and glosses, foundations, blushes, eyeliners, eyeshadows and the like. It will also be useful in treatment products, including pharmaceutical products, in which the stability of the antimicrobial is particularly crucial such as for example, ointments for wound cleansing, and the like.

Other biological active agents can be added to the structured water of the present invention or to the compositions containing the structured water as long as the presence of the silver ions and potassium sorbate in the cluster structure can be stabilized. The biological active agents are simply added after processing the feed water to produce the antimicrobial structured water or are added to compositions containing the structured water. The type of biological active agent added, can be any which is beneficially used in a topical cosmetic or pharmaceutical composition. For example, additional actives include but are not limited to, moisturizing actives, agents used to treat age spots, keratoses and wrinkles, as well as analgesics, anesthetics, anti-acne agents, antiyeast agents, antifungal agents, antiviral agents, antidandruff agents, antidermatitis agents, antipruritic agents, antiemetics, antimotion sickness agents, anti-irritant agents, anti-inflammatory agents, antihyperkeratolytic agents, anti-dry skin agents, antiperspirants, antipsoriatic agents, antiseborrheic agents, hair conditioners and hair treatment agents, antiaging agents, antiwrinkle agents, sunscreen agents, antihistamine agents, skin lightening agents, depigmenting agents, wound-healing agents, vitamins, corticosteroids, self-tanning agents, or hormones.

The following non-limiting examples illustrate the invention.

### EXAMPLES

### Example I

Antimicrobial Structured Water

| Ion | Amount (mg/100 ml) |
|---|---|
| CaCl(2) × 6 H(2)O | 10.00 |
| MgCl(2) × 6 H(2)O | 4.23 |
| Na(2)SO(4) | 5.00 |
| KH(2)PO(4) | 0.70 |
| KNO(3) | 1.00 |
| Potassium Sorbate | 40.00 |
| Silver Nitrate | 0.05 |

Feed water is prepared with the stabilizing cluster structure ionic composition described above by adding each ion to the feed water. After stabilizing ions are added, the potassium sorbate and silver nitrate are added to the feed water. The resulting feed water has a conductivity of about 450 to 550 μS/cm and a pH of about 6.0 to 6.5. The feed water is filtered through a tourmaline filter at a flow rate of about 200 L/hour, and then it is fed into the structured water making device at a flow rate of about 200 L/hour. The treated feed water is processed in a structured water producing device which has spaces for gathering and disposing the I water and S water. The spaces hold a volume of about 220 L. The dipolar molecular structure of the feed water containing silver ions and potassium sorbate is subjected to an electrostatic field having a voltage of about 80 V which causes the cluster structuring process. Negative Rₖ⁻ ions and negative ionic components of potassium sorbate (i.e., sorbate ions) are in the majority and the positive Rₘ⁺ ions (i.e., silver and potassium) are in the minority, and as a result of dissociation of the feed water containing the silver ions and potassium sorbate, they bind into clusters and migrate into the spaces for I water. The resulting I water has a pH of about 2.2 to 2.5 and a conductivity of about 1500 to 2200 μS/cm. The other result of dissociation produces S water where negative Rₖ⁻ ions are in the minority (i.e., the sorbate ions), and the positive Rₘ⁺ ions and positive ionic components of silver ions and potassium ions are in the majority. The resulting S water with mostly silver ions and potassium ions in its cluster structure has a pH of about 11.2 to 11.5 and a conductivity of about 1500 to 2000 μS/cm.

### Example II

Comparative Study

To demonstrate that the antimicrobial structured water of the present invention exhibits improved activity over simple addition of traditional antimicrobials to water, a comparative study is conducted. A sample of antimicrobial I and S water is compared with deionized water containing 40 mg/100 ml potassium sorbate, and the feed water used to make the I and S water. The feed water is prepared as described in Example I except that the feed water is not treated for cluster structuring for purposes of the comparative study. The deionized water containing 40 mg/100 ml potassium sorbate is prepared by simply adding potassium sorbate to the water, i.e., not treated to incorporate potassium sorbate into the cluster structure of structured water. Samples of antimicrobial I and S water samples prepared as described in Example 1. The test is a 10⁶ inoculation where each of five pools are inoculated with enterococcus, pseudomonas aeruginosa, staphylococcus aureus, yeast and mold respectively, for each of the four samples. The samples are reinoculated after 3 weeks and reinoculated again after 6 weeks. All five pools demonstrate antimicrobial activity for I water after the initial inoculation and the two subsequent reinoculations. Results indicate that S water exhibits activity against enterococcus, pseudomonas aeruginosa, and staphylococcus aureus, and therefore, demonstrates antibacterial activity. In comparison, however, four of the five pools of the feed water are contaminated after the first reinoculation, and demonstrate that the feed water with silver ions and potassium sorbate simply added to water, lack comparable antimicrobial activity. Finally, all of the pools of the deionized water containing 40 mg/100 ml potassium sorbate, failed to demonstrate antimicrobial activity. Moreover, these pools failed after the initial inoculation. Therefore, the antimicrobial activity of the I and S water of the present invention is due to the incorporation of silver ions and potassium sorbate in the cluster structure of the structured water.

### Example III

Makeup Remover Containing Antimicrobial Structured Water

| | |
|---|---|
| Ingredient | Percent |

| | |
|---|---|
| Antimicrobial Structured Water | 90.00 |
| Sucrose | 0.50 |
| Butylene glycol | 4.00 |
| Sodium chloride | 0.20 |
| Anti-irritant | 1.20 |
| Mild surfactant | 4.00 |
| Arginine | 0.10 |

This example illustrates the cosmetic or pharmaceutical composition containing structured water having silver ions and potassium sorbate in its cluster structure according to the present invention. When the silver ions and potassium sorbate are present within the cluster structure of structured water, the antimicrobial structured water is stable and does not succumb to the threat of instability due to external factors.

What we claim is:

1. A structured water comprising a cluster structure and at least two antimicrobial agents within said cluster structure.

2. The composition of claim 1 wherein one of said antimicrobial agents is a silver ion having a valency selected from the group consisting of one, two, and three.

3. The composition of claim 2 wherein one of said antimicrobial agents is potassium sorbate.

4. The composition of claim 1 wherein said cluster structure further comprises electronegative aggregates of water molecules forming I water.

5. The composition of claim 1 wherein said cluster structure further comprises electropositive aggregates of water molecules forming S water.

6. A structured water prepared by adding an antimicrobial effective amount of silver ions and potassium sorbate to an unstructured feed water, reducing the surface tension of the feed water, and processing the feed water in a device for producing structured water.

7. The structured water of claim 6 wherein said feed water has a pH of about 5.0 to 7.5 and a conductivity of about 350 to 550 μS/cm.

8. The structured water of claim 6 wherein the step of reducing the surface tension further comprises passing the feed water through a tourmaline filter.

9. A cosmetic or pharmaceutical composition containing the structured water of claim 1.

10. The composition of claim 9 wherein one of said antimicrobial agents is a silver ion having a valency selected from the group consisting of one, two, and three.

11. The composition of claim 9 wherein said structured water is selected from the group consisting of I water, S water, and a combination thereof.

12. The composition of claim 11 wherein said structured water is I water.

13. A topical cosmetic or pharmaceutical composition comprising the structured water of claim 6.

14. A method of producing structured water having antimicrobial activity comprising the steps of integrating silver ions and a stabilizing agent within a cluster structure of the structured water.

15. The method of claim 14 wherein the step of integrating the silver ions and the stabilizing agent within the cluster structure further comprises the steps of adding silver ions and the stabilizing agent to unstructured feed water, and processing the feed water in a device for producing structured water.

16. The method of claim 14 in which the stabilizing agent is potassium sorbate.

17. The method of claim 15 further comprising the step of reducing the surface tension of the unstructured feed water.

18. The method of claim 17 wherein the step of reducing the surface tension comprises passing the feed water through a tourmaline filter.

19. A method of ceasing or retarding the growth of bacteria comprising the step of applying the structured water of claim 1 to the skin.

20. A method of ceasing or retarding the growth of microbes comprising applying to the skin the structured water of claim 1.

21. A method of stabilizing silver ions having antimicrobial activity comprising the steps of preparing feed water containing 0.001 to about 1.0 mg/100 ml monovalent silver ions and 10 to 200 mg/100 ml potassium sorbate, passing the feed water through a tourmaline filter, and processing the tourmaline treated feed water in a structured water producing device.

22. A method of preserving a cosmetic or pharmaceutical composition comprising adding to the composition the structured water of claim 1.
